(19) United States
(12) Patent Application Publication
(10) Pub. No.: US 2012/0308989 A1
(43) Pub. Date: Dec. 6, 2012

(54) CONVERSION OF AQUATIC PLANTS TO LIQUID METHANE, AND ASSOCIATED SYSTEMS AND METHODS

(75) Inventors: John A. Barclay, Redmond, WA (US); David Haberman, Delray Beach, FL (US)

(73) Assignee: Prometheus Technologies, LLC, Redmond, WA (US)

(21) Appl. No.: 13/344,154

(22) Filed: Jan. 5, 2012

Related U.S. Application Data
(63) Continuation-in-part of application No. 12/792,653, filed on Jun. 2, 2010.
(60) Provisional application No. 61/429,991, filed on Jan. 5, 2011.
(60) Provisional application No. 61/183,516, filed on Jun. 2, 2009.

(51) Int. Cl. C12P 5/02; C12M 1/38; C12Q 3/00; C12M 1/107; C12M 1/36

(52) U.S. Cl. 435/3; 435/294.1; 435/286.6; 435/167

(57) ABSTRACT

Systems and methods for converting aquatic plants to liquid methane are disclosed. A representative system includes an aquatic plant cultivator, an anaerobic digester operatively coupled to the aquatic plant cultivator to receive aquatic plants and produce biogas, and a biogas converter coupled to the anaerobic digester to receive the biogas and produce liquefied methane and thermal energy, at least a portion of the thermal energy resulting from a methane liquefaction process. The system can further include a thermal path between the biogas converter and at least one of the aquatic plant cultivator and the anaerobic digester. A controller can be coupled to the biogas converter and the aquatic plant cultivator and/or the anaerobic digester. The controller can be programmed with instructions that, when executed (e.g., based on measured variables of the system), direct the portion of thermal energy between the biogas converter and the aquatic plant cultivator and/or anaerobic digester.

### CROSS-REFERENCE TO RELATED APPLICATIONS

The present application claims priority to U.S. Provisional Application No. 61/429,991, filed Jan. 5, 2011 and incorporated herein by reference. The present application is a continuation-in-part of pending U.S. application Ser. No. 12/792,653, filed Jun. 2, 2010 and incorporated herein by reference, which claims priority to U.S. Provisional Application 61/183,516, filed Jun. 2, 2009.

### TECHNICAL FIELD

Aspects of the present disclosure are directed to systems and methods for processing methane and other gases, including systems and methods for converting algae and/or other aquatic plants to liquid methane.

### BACKGROUND

Global warming and climate change are presently receiving significant scientific, business, regulatory, political, and media attention. According to increasing numbers of independent scientific reports, greenhouse gases impact the ozone layer and the complex atmospheric processes that re-radiate thermal energy into space, which in turn leads to global warming on Earth. Warmer temperatures in turn affect the entire ecosystem via numerous complex interactions that are not always well understood. Greenhouse gases include carbon dioxide, but also include other gases such as methane, which is about 23 times more potent than carbon dioxide as a greenhouse gas, and nitrous oxide, which is over 300 times more potent than carbon dioxide as a greenhouse gas.

In addition to the foregoing greenhouse gas concerns, there are significant concerns about the rate at which oil reserves are being depleted, and that the United States imports over 60% of the crude oil it consumes from a few unstable regions of the globe. Accordingly, there is an increasing focus on finding alternative sources of energy, including clean, renewable, less expensive, and domestic energy sources. These sources include municipal solid waste, food processing wastes, animal wastes, restaurant wastes, agricultural wastes, and waste water treatment plant sludge. These sources also include coal seam methane, coal mine gas, biomass, and stranded well gas. While many efforts have been undertaken to generate useable fuels from such sources, there remains a need to reduce the capital costs of fuel projects, to improve the efficiency with which such processes are completed, and to further reduce greenhouse gas emissions.

### BRIEF DESCRIPTION OF THE DRAWINGS

FIG. 1 is a schematic block diagram illustrating a representative system and method for converting algae and/or other aquatic plants to liquid methane via an anaerobic digester in accordance with an embodiment of the disclosure.

FIG. 2 is a partially schematic illustration of an aquatic plant raceway pond suitable for cultivating aquatic plants in accordance with an embodiment of the disclosure.

FIG. 3 is a partially schematic illustration of an anaerobic digester suitable for producing biogas in accordance with embodiments of the disclosure.

FIG. 4A is a flow diagram illustrating a system and method for liquefying biogas in accordance with an embodiment of the disclosure.

FIG. 4B is a partially schematic isometric illustration of a biogas converter having components for performing the methods shown in FIG. 4A.

FIG. 5 is a flow diagram illustrating a system and method for converting aquatic plants to liquefied methane in a closed loop fashion in accordance with an embodiment of the disclosure.

FIG. 6 is a schematic block diagram illustrating a system and method for converting aquatic plants to liquid methane in a closed loop fashion in accordance with another embodiment of the disclosure.

FIG. 7 is a schematic block diagram illustrating a system and method for converting aquatic plants to liquid methane in an open loop fashion in accordance with still another embodiment of the disclosure.

### DETAILED DESCRIPTION

Several aspects of the present disclosure are directed to systems and methods for processing methane and other gases. Well-known characteristics often associated with these systems and methods have not been shown or described in detail to avoid unnecessarily obscuring the description of the various embodiments. Those of ordinary skill in the relevant art will understand that additional embodiments may be practiced without several of the details described below, and/or may include aspects in addition to those described below.

### Overall System

Aspects of the present disclosure include the combination of three dissimilar technologies to create new processes and associated systems for the purpose of economically transforming microalgae and/or other aquatic plants blended with municipal solid waste or other suitable organic material into liquid natural gas (LNG) or liquid biomethane (LBM), collectively referred to as “LNG/LBM” or “liquid methane.” These three technologies include aquatic plant growth, anaerobic digestion of an aquatic plant biomass to make biogas, and purification/liquefaction of biogas to make low cost LNG/LBM. In particular embodiments, multiple operational challenges (including thermal management, nutrients balance, and water recycling) can be met by integrating the foregoing technologies. To efficiently grow generic aquatic plant species, systems and methods in accordance with embodiments of the disclosure use carbon dioxide feed stock, nutrients, water, and sunlight. To efficiently convert the aquatic plants into biogas, systems and methods in accordance with embodiments of the disclosure include blending an aquatic plant biomass with organic waste streams including paper and/or municipal solid waste. The high quality LNG/LBM made from the methane-containing biogas can be stored, transported, and distributed for use as cleaner, domestic, more economic, and renewable vehicle fuel in the transportation sector or as the fuel of choice in other energy sectors. In certain embodiments, the system includes a unified facility that simultaneously captures carbon dioxide, processes multiple organic waste streams, and produces high quality, low cost liquefied biomethane for vehicular fuel. Accordingly, it is expected that embodiments of the systems and methods disclosed herein will directly reduce greenhouse gas emissions and significantly reduce oil imports.

A representative system includes a facility that simultaneously enables distributed-scale, high productivity aquatic plant cultivation and its conversion to biogas and LNG/LBM in locations with high solar input but thermally extreme climates. The carbon dioxide feedstock required for efficient aquatic plant bioreactors may be received from any of a variety of concentrated sources, including flue gas. A further feature of an embodiment of the system includes a modular, portable digester and a modular, portable purifier/liquefier for converting digester biogas into LNG/LBM in accordance with two different types of aquatic plant cultivation schemes; one in a warm dry climate (e.g., the Arizona desert) and the other at a warm humid climate (e.g., the Southeastern United States). These modular, portable systems can be connected using innovative arrangements to make and sell heavy duty vehicular fuel at substantially less cost than present diesel fuel, and simultaneously provide reduced greenhouse gas emissions.

Particular aspects of the present technology are described below in the context of algae cultivation and converting algae to biogas. In other embodiments, similar techniques are used to produce LNG/LBM from other aquatic plants (e.g., other aquatic, photosynthesizing organisms), including but not limited to duckweed. Many of the aspects of the technology described below in the context of algae are therefore applicable to other aquatic plants as well. Specific instances in which the techniques are different for algae than they are for aquatic plants other than algae are highlighted below. Other features that are not highlighted are expected to be applicable to both algae and other aquatic plants.

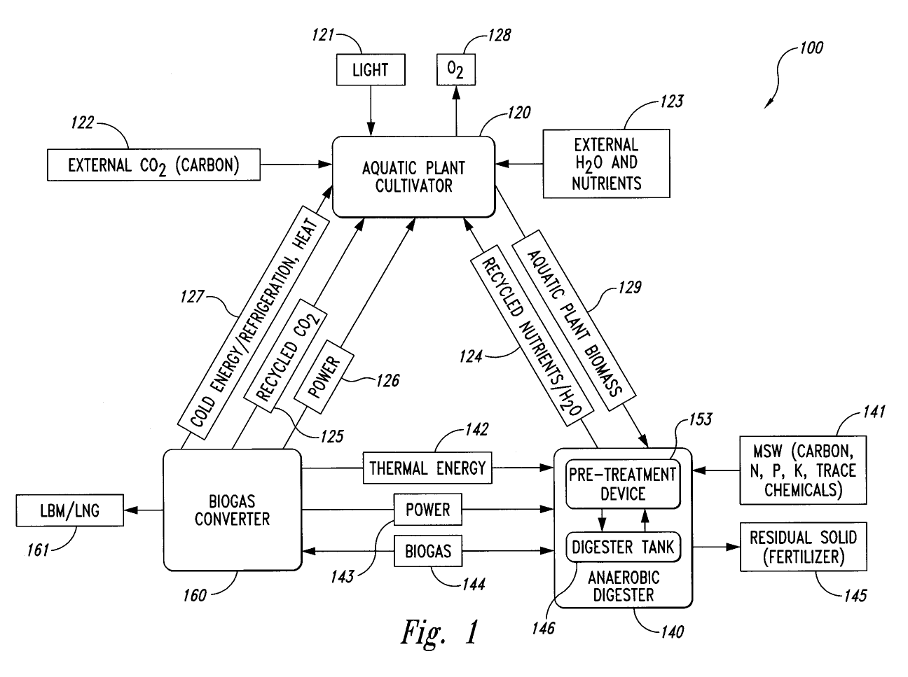

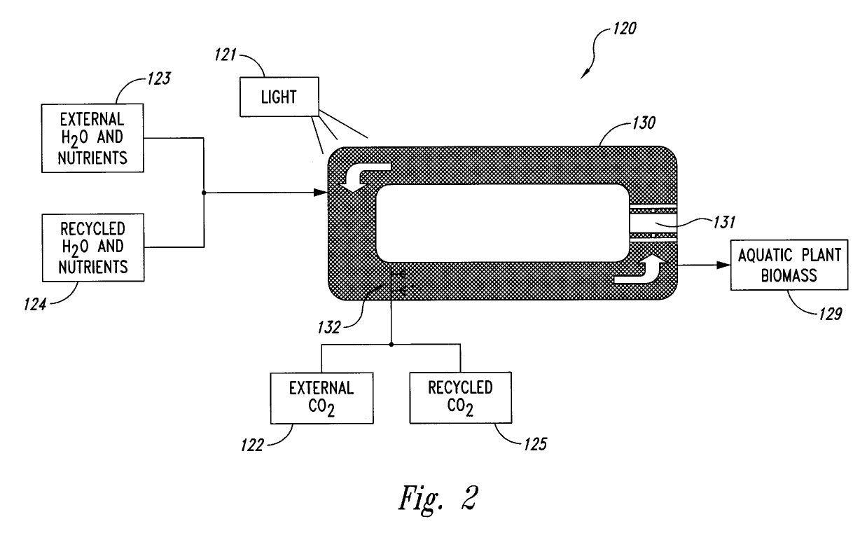

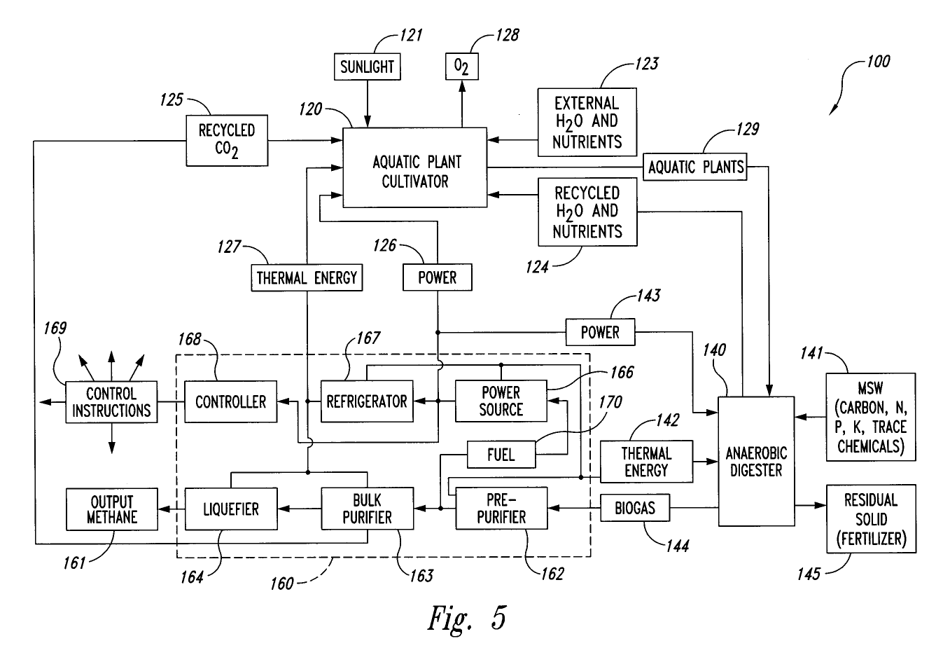

FIG. 1 is a schematic block diagram illustrating the overall operation of a system 100 in accordance with an embodiment of the disclosure. The system 100 can include three basic elements that can work in combination in a batch, continuous flow, or mixed batch/continuous flow process to produce liquid methane. The three basic elements include an aquatic plant cultivator 120, an anaerobic digester 140, and a biogas converter 160. The general relationships between these system components are described further below with reference to FIG. 1. Specific aspects of each of these components are then described further with reference to FIGS. 2-4B. FIGS. 5-7 describe integrated systems in accordance with several embodiments of the disclosure.

As shown in FIG. 1, the aquatic plant cultivator 120 includes a suitable facility for growing aquatic plants and accordingly requires inputs to support this biological process. The inputs can include light (e.g., sunlight 121), external carbon dioxide 122, (e.g., from air and/or liquefied or solid CO₂), and external water and nutrients 123. In particular embodiments, the external carbon dioxide 122 and external water and nutrients 123 (e.g., nitrogen, phosphorus, and potassium) can be supplemented or replaced by recycled constituents from other portions of the system 100. For example, the aquatic plant cultivator 120 can receive recycled carbon dioxide 125 from the biogas converter 160, and can receive recycled nutrients (e.g., nitrogen, phosphorus, and potassium) and water 124 from the anaerobic digester 140. In particular embodiments, the aquatic plant cultivator 120 can also receive thermal energy 127 (e.g., cold energy/refrigeration, and/or heat) from the biogas converter 160 to regulate the temperature of the aquatic plants. By regulating the temperature of the aquatic plants, the aquatic plant growth rate can be optimized or at least enhanced. The aquatic plant cultivator 120 can receive power 126, also from the biogas converter 160 to power certain features of the aquatic plant cultivator 120 as described further below. In any of these embodiments, the aquatic plant cultivator 120 can output oxygen 128 and an aquatic plant biomass 129. The oxygen 128 can be captured for other uses, or released into the local environment. The aquatic plant biomass 129 can be provided to the anaerobic digester 140. Certain species of microalgae biomass and/or other aquatic plants can also be used as feedstock for food additives or for medical uses.

The anaerobic digester 140 can include a pre-treatment device 153 and a digester tank or vessel 146 that may allow protein extraction from a portion of the biomass for food by-products. The pre-treatment device may allow easy collection of algae or other aquatic plant biomass, mixing of biomass sources, and heating for sterilization of combined biomass to augment anaerobic digestion. At the anaerobic digester 140, the aquatic plant biomass 129 is bacterially converted or otherwise processed to produce an output biogas 144 that contains methane. By-products 145 can be used for fertilizers or other purposes. In particular embodiments, the anaerobic digester 140 can also receive external waste 141, for example, paper, animal manures, municipal solid waste, or other sources of feedstock with selected compositions of carbon, nitrogen, phosphorous, potassium, and/or other trace chemicals to supplement the aquatic plant biomass 129 received from the aquatic plant cultivator 120 for optimal or otherwise enhanced digestion by anaerobic bacteria consortia. The anaerobic digester 140 and the pre-treatment device 153 can, in particular embodiments, receive thermal energy 142 and/or power 143 from the biogas converter 160. The thermal energy 142 can be used to keep the anaerobic digester 140 within a target range of temperatures selected to produce a high output rate of the biogas 144. The power 143 received at the anaerobic digester 140 can be used to power certain components of the anaerobic digester 140, as described later. In any of these embodiments, the biogas 144 can include a significant methane component. To improve the utility of the biogas 144, the biogas converter 160 can be used to purify and convert the biogas 144 into liquid biomethane.

At the biogas converter 160, the biogas 144 received from the anaerobic digester 140 is compressed, purified (e.g., to remove water and carbon dioxide) and liquefied (e.g., to provide a suitable fuel for heavy duty transportation vehicles), resulting in output methane 161. The removed carbon dioxide can be used at least in part to form the recycled carbon dioxide 125 provided to the aquatic plant cultivator 120. A portion of the cold energy or refrigeration produced within the biogas converter 160 (which is generally used to liquefy the methane) can additionally or instead provide the cold energy or refrigeration 127 used by the aquatic plant cultivator 120 during periods when the solar energy increases the water temperature above that for optimal aquatic plant growth. If the temperature of the aquatic plant cultivator 120 decreases below that for optimal aquatic plant growth, the aquatic plant cultivator 120 can receive heat from the biogas converter 160. In addition, the biogas converter 160 can include a power generator (e.g., a genset) that provides electrical power 126, 143 to the aquatic plant cultivator 120 and the anaerobic digester 140. In at least some embodiments some of the methane in the biogas is burned to produce the power 126, 142. The waste thermal energy from the power generator can be transferred between the biogas converter 160 and other system components (e.g., the aquatic plant cultivator 120 and/or the anaerobic digester 140) to more efficiently process the methane produced by the biogas converter 160. The thermal energy exchange from the biogas converter 160 to the aquatic plant cultivator 120 and/or the anaerobic digester 140 can be accomplished by a suitable circulating heat transfer fluid, which can include carbon dioxide as discussed above, or other fluids in other embodiments. The output methane 161 produced by the biogas converter 160 can include liquid biomethane, liquid natural gas, or a combination of the two. This output product can be provided directly to transportation vehicles or other end use applications at the biogas converter 160, or the output product can be shipped by truck, rail, ship, pipeline or other suitable methods to distribution sites located remotely from the facility. In general, the output methane is primarily in the form of LNG/LBM but a portion of it may also be in other forms such as LCNG for light duty vehicle fuel in local vehicles or PNG that is injected into a local pipeline that may be located near this plant site. In any of these embodiments, the integrated operation of the aquatic plant cultivator 120, the anaerobic digester 140, and the biogas converter 160 can improve the efficiency with which the output methane 161 is produced, and can reduce the carbon footprint of the process by internally recycling intermediate products.

### Aquatic Plant Cultivator

Microalgae are among several types of aquatic plants that convert carbon dioxide, water, nutrients, and light (e.g., solar energy) into biomass via photosynthesis. Factors that influence photosynthetic efficiency include the irradiance and wavelength of the light, the carbon dioxide concentration, and temperature. The complex process creates carbohydrates, lipids, and proteins, and releases oxygen. There are approximately 200,000 or more species of algae that produce approximately 50% of the earth's oxygen. The algae themselves are approximately 50% carbon. Because microalgae have very high specific areas (surface area per unit volume), they can rapidly uptake nutrients and carbon dioxide and typically grow much faster than land-based plants.

Growing algae efficiently typically requires sunlight, water, carbon dioxide, and other nutrients, primarily nitrogen and phosphorus with other trace elements, such as silicon, iron and magnesium. Growth rates can be extremely high; in practice they vary substantially among species and conditions, with 20-30 gm/m²/d [grams of algae biomass per square meter per day] being a reasonable average value of production in stirred open ponds or closed bioreactors. Such growth rates are expected to use 40-60 gm of CO₂/m²/d as carbon nutrient input.

FIG. 2 is a schematic illustration of a portion of an aquatic plant cultivator 120 configured in accordance with an embodiment of the disclosure. In this particular embodiment, the aquatic plant cultivator 120 includes one or more raceway ponds 130 in which the algae grows. The raceway ponds 130 can be configured to produce a large amount of algae with a relatively small amount of surface area and water. For example, a representative raceway pond 130 can have a depth of about 10 inches so as to concentrate algae growth in the region of the water most likely to be penetrated by the sunlight 121. Water and nutrients (e.g., the external water and nutrients 123 and/or recycled water and nutrients 124) are provided to the raceway pond 130 via a suitable intake. The raceway pond 130 receives external carbon dioxide 122 and/or recycled carbon dioxide 125 via a sparger 132 or other introducer throughout the water depth, or near the surface of the water depending upon whether the aquatic plant is a microalgae that is distributed below the surface, or a floating species such as duckweed. A mixer 131 (e.g., a paddle or arrangement of low power fluid pumps) slowly circulates the constituents in the raceway pond 130 to increase the uniformity with which the constituents are distributed. The mixer 131 can be powered by energy generated at the biogas converter 160, as described above with reference to FIG. 1, or it can be powered by another source such as a solar panel. In any of these embodiments, the raceway pond 130 or a network of raceway ponds 130 can produce the aquatic plant biomass 129 used by the anaerobic digester 140 and for food by-products in particular embodiments (FIG. 1).

In particular embodiments, the carbon dioxide feedstock is sparged into the water to create concentrations near the limit of carbon dioxide solubility in water, and much higher than the typical concentration of carbon dioxide in air (which is about 375 ppm). In a particular embodiment, a portion of the carbon dioxide feedstock (e.g., about 20%) comes from the biogas converter 160 (FIG. 1) and the rest from external sources. Suitable external sources include industrial sources (e.g., captured flue gas from power plants, and/or bulk carbon dioxide produced at landfill gas-to-LNG plants) among others. The external carbon dioxide can be effectively delivered as a chilled liquid via insulated tankers, as a gas via pipelines, and/or as solid dry ice via insulated trucks or other suitable transportation systems (e.g., an insulated conveyer or other feed system). If the carbon dioxide is sufficiently cooled, e.g., as a liquid or a solid, it can be used for thermal management of the aquatic plant cultivator 120 as well. For example, dry ice (e.g., crushed or small pellets) can be distributed into the raceway pond 130 via several spargers 132 not only to provide carbon dioxide to the algae and/or other water plants, but also to cool the pond 130 via latent heat of sublimation and sensible heat of the carbon dioxide. In another embodiment, the carbon dioxide can be warmed using the waste gas from the genset before injecting it into the aquatic plant cultivator 120 during the colder months of the year. This arrangement can keep the conditions in the raceway pond 130 within a temperature range expected to produce large quantities of the aquatic plant biomass 129. An advantage of this arrangement is that the carbon dioxide can perform two control functions simultaneously, while recycling waste from the biogas converter 160. These functions can be selectively controlled independently, for example, if the need for carbon dioxide at the aquatic plant cultivator 120 does not precisely align with the need for cooling or heating. In a particular embodiment, as discussed above, a portion of the refrigeration capacity of the biogas converter 160 can be used to cool the aquatic plant cultivator 120. In other embodiments, the waste thermal energy from the biogas converter 160 can be used to warm the cultivator or alternatively, generate refrigeration, e.g., via an absorption cooler or a heat engine-driven refrigerator. The excess heat can include low grade heat (e.g., 200° F.) resulting from the compressors in the refrigeration cycle used to liquefy the methane, and/or high grade heat (e.g., 900° F.) produced as waste by the genset or other power production device. In still further embodiments, the aquatic plant cultivator 120 can include a distributed heat exchanger structure, e.g., for use when carbon dioxide is not the heat transfer media.

In a particular embodiment, the nitrogen and phosphorus nutrients described above are chemically bound into the protein fraction of the aquatic plant biomass. The nitrogen, phosphorus, and potassium feedstock inputs and the water for the aquatic plant cultivator 120 can be supplied primarily by a suitably operated anaerobic digester 140 or from external sources (FIG. 1).

The aquatic plant cultivator 120 can be sited at any of a variety of suitable locations that provide access to the ingredients used for rapid plant growth. These include a suitable amount of land or other surface area, a suitable amount of carbon dioxide and other nutrients, plentiful sunlight, and moderate temperatures over a suitable portion of the year. A representative temperature range is 25-30° C. [77-86° F.] during sunlight hours, and lower temperatures at night, as algae growth drops off sharply when the temperature increases above about 95° F. or falls below about 45° F. Representative sites include those found in the southern one-third of United States, and other climactically similar locations around the world. Such locations can be located within a latitude band of +30° from the equator.

As shown in FIG. 2, the aquatic plants may be grown in an open raceway pond. This arrangement can be used, for example, in the south/southeastern United States, where the yearly average irradiance is approximately 195 W/m². In the hottest months of the year the, water evaporation from these ponds is relatively small because of the extremely high relative humidity, and accordingly, the ponds need not be enclosed.

In other embodiments, the aquatic plants can be grown in other facilities. One such facility is a closed photobioreactor, which can have particular utility in the south/southwestern United States where the average annual irradiance is approximately 225 W/m². Closed photobioreactorsare suitable in such areas because water evaporation is large due to low relative humidity. In particular closed photobioreactors, carbon dioxide is sparged into the flowing aquatic plant/water/nutrient mixture along the entire flow path of the reactor.

In general, aquatic plant photosynthesis only uses about 5% of the incident solar insolation. Much of the remaining incident energy is converted to heat. Excess thermal energy in the southwestern United States during the summer months can accordingly create a dynamic thermal management challenge for closed photobioreactors. For example, the water temperature in a closed photobioreactor can increase from about 25° C. to about 42° C. or 108° F., even with stirring. Open raceway ponds can also provide thermal management challenges, though the peak temperature may not be as high. As will be described in further detail later, the aquatic plant biomass is concentrated as part of the pre-treatment process and as it is blended with the municipal solid waste stream in the anaerobic digester system. No special flocculation technique or dewatering/drying of the aquatic plant biomass 129 is required. Accordingly, collecting and concentrating the algae and/or other aquatic plants can be more efficient and less expensive than conventional techniques.

To address the foregoing (and/or other) thermal management challenges, embodiments of the present disclosure can include one or more of several integrated cooling techniques. Suitable techniques include controlled evaporation, ground or air circulation loops to reject heat to ambient, and/or active cooling, e.g., vapor compression cycle or advanced refrigerator cooling, including via a magnetic refrigerator. As described above, cooled carbon dioxide can be used in addition to or in lieu of the foregoing techniques. In addition to or in lieu of cooling, rejected heat from the biogas converter 160 (e.g., transferred via heated carbon dioxide) can be used to heat the aquatic plant cultivator 120 if temperatures drop significantly. Accordingly, the transfer of thermal energy between the biogas converter 160 and the aquatic plant cultivator 120 can operate to cool or heat the aquatic plant cultivator 120, depending upon the temperature at the aquatic plant cultivator 120.

Because the aquatic plant biomass output from the aquatic plant cultivator 120 plant is transferred to the anaerobic digester 140 (FIG. 1) in total, when algae is the selected aquatic plant, multiple species of algae can be used in the cultivator 120. In particular, there is no need to select or maintain algae species that have a high lipid content. As a result, the aquatic plant cultivator 120 can provide a degree of robustness that is generally not associated with algaes produced for biodiesel fuels. For example, numerous naturally occurring algae species can be cultivated using the presently disclosed technology, as opposed to using more expensive genetically modified species.

As noted above, the aquatic plants cultivated in the aquatic plant cultivator 120 can include algae (e.g., microalgae) in particular embodiments, and can include other plants in other embodiments. In general, the plants grown in the aquatic plant cultivator 120 have rapid growth rates, are easily harvested, and are easily digested at the anaerobic digester 140 described in greater detail below. A particular example of a suitable aquatic plant is duckweed, e.g., plants in any of the genea lemna, spriodela, wolfia, and wolffiella. Duckweed is generally a floating plant, and has a rapid growth rate roughly equivalent to that of microalgae. Duckweed can be grown in layers (e.g., up to about six or more layers) which are generally stacked directly upon each other at the surface of the water. The duckweed can be harvested by arranging a suitable suction plate just below the surface of the water in order to harvest the bottom layers (e.g., the bottom two to three layers) of duckweed, without removing the remaining layers above. Additional layers of duckweed will subsequently grow on top of the remaining layers, forcing the remaining layers downward in the stack where they will be subsequently removed.

Carbon dioxide can be supplied to the duckweed or other aquatic plant in a manner generally similar to that described above, by sparging dry ice into the water, e.g., just below the depth where the sunlight is significantly reduced (e.g., by about 90%) from that available on the surface of the pond. The dry ice particles can move slowly with the circulating water in the aquatic plant cultivator. As each particle sublimes into gaseous carbon dioxide, it will rise to the surface of the water. Because gaseous carbon dioxide is denser than air, the carbon dioxide will tend to remain at or near the surface of the water, which can be particularly suitable for enriching the carbon dioxide environment adjacent to floating plants, such as duckweed.

In particular embodiments, the carbon concentration can be further enhanced by covering or otherwise enclosing the aquatic plant cultivator 120 e.g., forming an enclosed photobioreactor. The extent to which such an enclosure is require can depend at least in part on whether the environmental conditions allow the carbon dioxide released from the dry ice to remain close to the water's surface. Such enclosures may be used, for example, where the local winds are strong enough to blow the gaseous carbon dioxide away. In another embodiment, low barriers, baffles, and/or other impediments can be used to shield the water's surface from winds, without necessitating the expense and complexity of a roofed enclosure.

### Anaerobic Digester

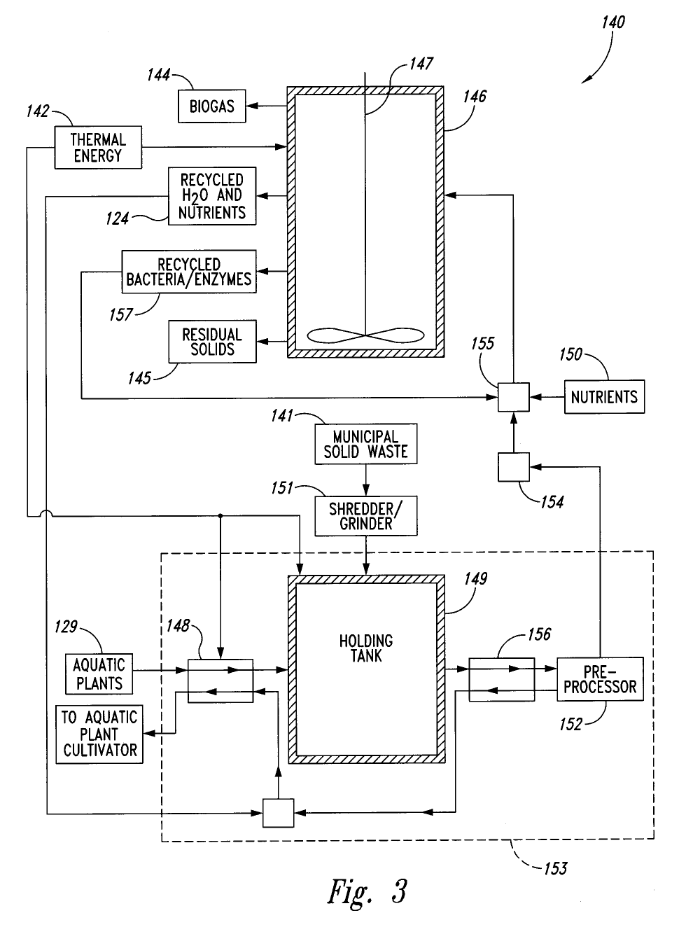

FIG. 3 is a block diagram illustrating features of an anaerobic digester 140 configured in accordance with a particular embodiment of the disclosure. The digester 140 can include a digester tank or vessel 146 (with various consortia of bacteria) that anaerobically converts an organic feedstock to biogas having between 65% and 75% methane, with the remainder being largely carbon dioxide. Up to 85% of the volatile solids are converted to biogas (under selected, e.g., optimal conditions) and the residual can provide a rich fertilizer product. In particular embodiments, the digester tank 146 can include internal jet-type pumps or a mixer 147, and can have an insulated stainless steel construction up to a capacity of about 750,000 gallons to one million gallons. The digester tank 146 can process volatile organic solids in a range of concentrations in water of about 5% to about 15% by volume and in a particular embodiment, about 10%. The capacity of the digester 140 can be increased by adding additional tanks 146 in parallel to feed a common biogas header. In a particular embodiment, 6-7 tanks can be provided at a facility that produces about 20,000 gpd net of LBM. The large capacity tanks result in part from the fact that in at least some embodiments, the aquatic plant biomass is supplemented with municipal solid waste, which in at least some cases, can almost double (a) the amount of biogas produced at the anaerobic digester 140, and (b) the output of LNG/LBM.

The composition of the aquatic plant biomass 129 entering the digester tank 146 can be a significant design factor for the digester 140. In particular embodiments, the average composition of an algae biomass is CO_{0.48}H_{1.83}N_{0.11}P_{0.01} with proteins [C₆H_{13.1}O₁N_{0.6}] ranging from 6-52% depending upon the species; lipids [C₅₇H₁₀₄O₆] ranging from 7-23% with a few selected species being as high as about 50%; and carbohydrates [C₆F₁₀O₅]ₙ ranging from 5-23% again depending on the species. The average carbon/nitrogen (C/N) ratio for an algae biomass is approximately 10 for freshwater microalgae, a sharp contrast relative to a typical terrestrial plant biomass, for which the C/N ratio can be as high as about 36. To increase the C/N ratio of a microalgae-based biomass-water mixture provided to the digester tank 146, waste streams from different external waste sources can be mixed with the dilute algae biomass-water stream. This arrangement can raise the C/N ratio to 20-25 while providing the proper nitrogen, phosphorous and potassium nutrient balance within the digester tank 146 to achieve suitable/optimal conditions for the consortia of bacteria and enzymes in the tank 146. Waste paper and/or municipal solid waste (MSW) provide appropriate sources, and the income received from processing MSW or other waste streams can be a significant revenue source for the overall system 100 (FIG. 1). In particular embodiments, the waste is received from local sources so as to reduce the expense and carbon footprint associated with transporting these materials. The additional biogas provided by these additional waste streams also increases the amount of LNG/LBM from the overall system 100.

Duckweed and other non-algae aquatic plants may have a C/N ratio higher than that for microalgae. For example, duckweed is expected to have a C/N ratio of about 30 after proteins from the duckweed are extracted. The proteins can be extracted using a lycing process in a rotating ball mill with progressively smaller sized balls that break the duckweed cell walls and separate the protein and the carbohydrate portions of the duckweed. The proteins can be used to make food products, thus providing an additional revenue stream for the overall system. Given the increased C/N ratio of duckweed, the need for MSW or other waste streams can be reduced or in some cases, eliminated. In particular embodiments, microalgae and duckweed can be grown at the same facility (but in different ponds) and then mixed to produce the desired C/N ratio.

The temperature of the digester tank 146 is also important for either mesophilic or thermophilic consortia of anaerobic bacteria. Mesophilic or lower temperature consortia are more tolerant of temperature variations but still require controlled temperatures of about 35° C. [95° F.], while thermophillic consortia require controlled temperatures of about 55° C. [131° F.]. One feature of an embodiment of the digester 140 shown in FIG. 3 is that the waste thermal energy 142 output from the biogas converter 160 (FIG. 1) is input to the digester 140 to maintain the temperature in the digester tank(s) 146 within the close tolerances that provide for rapid digestion. The average amount of biogas produced varies with the components of the aquatic plant biomass. When microalgae is selected as the aquatic plant, the components can include proteins at about 0.85 liter (L) CH₄/gm VS [volatile solids]; lipids at about 1.01 L CH₄/gm VS; and carbohydrates at about 0.45 L CH₄/gm VS. An example is Chorella vulgaris with 51-58% protein, 14-22% lipid, and 12-17% carbohydrate makes 0.63-0.70 L CH₄/g VS. The typical digester biogas produced will have a composition of 65±5% CH₄, 35±5% CO₂, 1000 ppmv N₂, 10 ppmv O₂, 1000 ppmv H₂S and other VOCs, no siloxanes, 2±1% H₂O (saturated), a pressure of about 1 psig, and a temperature ranging from about 95° F. to 130° F., depending on the type of consortia selected.

Another feature of an embodiment of the digester 140 shown in FIG. 3 relates to collecting and pre-processing the aquatic plant biomass stream as it is sent to the digester tank 146. The cell walls of microalgae (and/or other aquatic plants) resist anaerobic bacteria/enzyme attack which inhibits the production of biogas. The aquatic plants are also in relatively low concentrations in the cultivator water and must be concentrated for optimal digester operation. To address these aspects in accordance with a particular embodiment of the disclosure, the aquatic plant biomass is collected without adding flocculation agents. Instead, the aquatic plant biomass 129 can include a flow of aquatic plant-loaded water that is diverted from the cultivator through a first heat exchanger 148 (e.g., a counterflow heat exchanger) where it can be heated before it goes into a heated holding tank or vessel 149, e.g., having a size similar to that of one of the digester tanks 146. Depending on the C/N ratio of the aquatic plant biomass 129, a controlled amount of MSW 141 or other high C/N ratio waste can also be supplied to the holding tank 149, after passing through a shredder/grinder 151. For example, the incoming stream of aquatic plant biomass 129 (at about 25° C.) can be provided to the heat exchanger 148, where it is heated (e.g., to about 80° C.-90° C.) by water and nutrients removed from a downstream pre-processor 152 and/or by thermal energy 142 received from the biogas converter 160 (FIG. 1) such that the entire contents is at an elevated temperature.

The elevated temperature in the holding tank 149 is maintained for several hours to eliminate pathogens and/or undesirable bacteria and/or other constituents that may inhibit the anaerobic digestion process. In particular embodiments, the holding tank 149 is insulated and/or heated (e.g., with waste heat 142 from the biogas converter 160) to maintain a suitable temperature. In a representative embodiment, the contents of the holding tank 149 can be held for a period of about 8 hours at a high temperature (e.g., 80° C.) to kill the aquatic plants and begin to break down their cell walls. The elevated temperature can also kill pathogens in the aquatic plant stream and/or the MSW stream. The dead aquatic plants and sterilized waste settle under gravity toward the bottom of the holding tank 149 and can be pumped into the pre-processor 152.

At the preprocessor 152, the moisture content of the combined waste stream can be adjusted by suitably meshed filters. For example, the solid fraction of the stream can be adjusted to about 10% volatile solids for suitable operation of the digester tank 146. In a typical process, the mixture enters the pre-processor 152 with a solid content lower than 10% (e.g., 2-3%) and so adjusting the water content includes removing liquid from the mixture. The concentrated mixture (e.g., of aquatic plants and MSW) can then be cooled to approximately the temperature desired within the digester tank 146. In a particular embodiment, a second heat exchanger 156 cools the incoming stream with water withdrawn from the pre-processor 152. After passing through the second heat exchanger 156, the withdrawn water (now heated), is used to heat the aquatic plant stream at the first heat exchanger 148. Once the solid fraction of the flow is properly adjusted at the pre-processor 152, it is pumped away from the pre-processor 152 via a pump 154. The flow can be inoculated with anaerobic bacteria and enzymes 157, which may be removed from the digester tank 146 or obtained from other suppliers and mixed with the flow at an innoculator 155. Optionally, additional nutrients 150 can also be added to the flow before the flow is provided to the digester tank 146.

At the digester tank 146, the flow is further mixed and anaerobically processed to produce the biogas 144, which is then provided to the biogas converter 160 (FIG. 1). The digester tank can be insulated and can optionally be heated, e.g., via waste heat 142 from the biogas converter 160. Byproducts from the digestion process include the residual solids 145 (e.g., sold for fertilizer) and aquatic plant-free water/nutrients 124 (e.g., water and nitrogen, nitrates, phosphorus, and/or potassium (potassium nitrate and/or ammonium nitrate)). The water/nutrients 124 can be routed through the first heat exchanger 148, as discussed above, to cool the water/nutrients 124 before they are returned to the aquatic plant cultivator 120. This arrangement can be particularly suitable in the context of algae processing. When the potassium, nitrates and phosphorus are removed from the stream in the form of proteins to form food products, as discussed above in the context of duckweed, these nutrients will typically be replenished at the aquatic plant cultivator via a separate process.

An advantage of the foregoing arrangement is that it can reduce or eliminate the need to flocculate the aquatic plants, which adds expense to the overall process. Instead, the aquatic plant stream can be processed by using existing waste heat to kill the aquatic plants, and then remove water from the aquatic plant stream.

### Biogas Converter

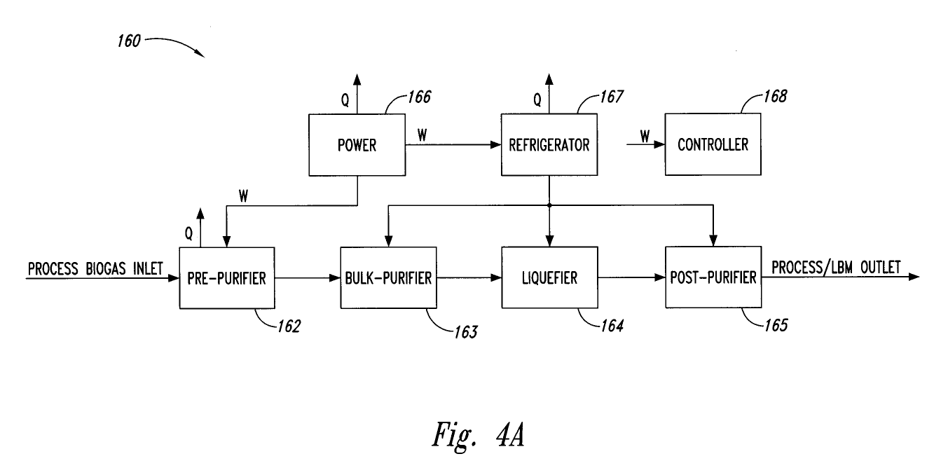

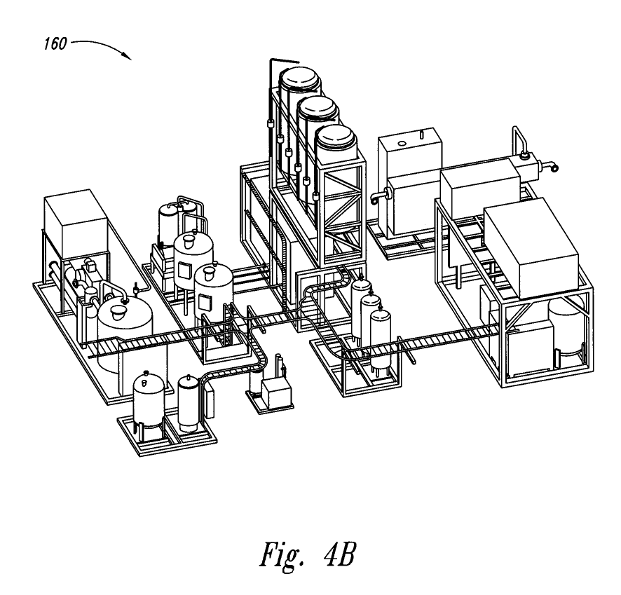

FIG. 4A is a schematic illustration of a representative biogas converter 160 for purifying and liquefying a stream of process gas in accordance with a particular embodiment of the disclosure. The illustrated converter 160 receives an input stream of gas (e.g., biogas) and produces a liquefied product (e.g., liquefied methane). The converter 160 can include a pre-purifier 162, a bulk purifier 163, a liquefier 164 driven by a refrigerator 167, and a post-purifier 165. In other embodiments, the converter 160 can include more or fewer modules. For example, in many cases, the post-purifier 165 can be eliminated because the amount of N₂ gas resulting from the process can be very low. In any of these embodiments, a power source 166 can provide work/power (indicated by arrow W) to operate the modules of the converter 160 and/or other components of the overall system 100 described above with reference to FIG. 1. Several of the foregoing modules release heat (indicated by arrows Q) which can either be discharged, or, as discussed above, used by other components of the overall system 100. A controller 168 controls the operation of the modules, with or without intervention by a human operator, depending on the phase of operation. Other aspects of the converter 160 in accordance with particular embodiments of the disclosure are included in U.S. Pat. No. 6,082,133, incorporated herein by reference, and pending U.S. Application Publication No. 2008/0289497, also incorporated herein by reference.

FIG. 4B is a schematic illustration of a converter 160 operated by the assignee of the present invention at a landfill site to convert landfill gas (LFG) to liquid natural gas (LNG). Many aspects of the illustrated system may also be used to convert biogas to LBM and/or LNG. The illustrated system has a targeted maximum capacity of approximately 4,500 gpd of high quality LNG using approximately 1.3 MMscfd of LFG containing approximately 48% methane. The system can include several modules (e.g., skid-mounted equipment) for converting the dirty LFG into high quality LNG. These modules can be placed in corresponding ISO containers so as to be moved among different sites, for testing and/or production. The containers can shield the components from environmental elements, reduce the need for support pads, and/or improve mass producability. The modules can include a pre-purification module (corresponding to reference number 162 in FIG. 4A), designed to remove water, sulfur compounds, and non-methane organic compounds (NMOCs) from the LFG process stream and compress the partially purified LFG from about 15 psia to about 125 psia. Another module (corresponding to reference number 163 in FIG. 4A) provides for bulk carbon dioxide removal. This module can remove carbon dioxide in two steps; by directly freezing out the carbon dioxide and by temperature swing adsorption. Accordingly, the module can extract carbon dioxide from the incoming biogas in a manner that (a) produces a purified methane stream, (b) produces carbon dioxide for use by the aquatic plant cultivator 120, and (c) recycles waste thermal energy resulting from generating power with a portion of the methane. Still another module (corresponding to reference number 167 in FIG. 4A) provides refrigeration. This module can provide cryogenic cooling using high purity nitrogen gas as the refrigerant in a closed refrigeration cycle. In other embodiments, this module can provide cryogenic cooling using a low pressure (e.g., peak pressure of about 300 psi) mixed refrigerant cycle for which the refrigerant can include a mixture of two or more of the following constituents: iso-pentane, n-butane, propane, ethane, ethylene, methane, argon and nitrogen. A liquefaction and post-purification module (corresponding to reference numbers 164 and 165 in FIG. 4A) provides liquefaction and post purification. This dual purpose module can liquefy the pre-cooled methane process stream, collect the liquid, and then send the liquid biomethane to an insulated storage tank. The post-purification portion of this module may not be needed for processing biogas from the anaerobic digester 140 (FIG. 1) because (as discussed above) such gas typically has little nitrogen. The fuel for the power generator can be extracted as a slip stream from partially purified biogas.

A control module (corresponding to reference number 168 in FIG. 4A) provides controls, including power distribution panels, instrumentation, and operator interfaces. A power module (corresponding to reference number 166 in FIG. 4A) provides system power. In a particular embodiment, the power module includes a natural gas driven genset with a maximum capacity of about 1.06 MW of electrical power. Still further modules can include an LNG storage tank, and a truck scale and LNG transfer system to load LNG from the storage tank into a cryogenic tanker.

### Integrated Systems

FIG. 5 is a schematic block diagram illustrating an embodiment of the system 100, with aspects of the biogas converter 160 described above with reference to FIGS. 4A-4B integrated with aspects of the aquatic plant cultivator 120 and the anaerobic digester 140 described above with reference to FIGS. 1-3. Accordingly, FIG. 5 illustrates the biogas converter 160 providing thermal energy 127 (heat and/or refrigeration) and power 126 to the aquatic plant cultivator 120, and providing power 143 and thermal energy 142 to the anaerobic digester 140. Pre-purified biogas 170 (primarily methane) is used to drive the power source 166. The controller 168 of the biogas converter 160 can provide control instructions 169 that direct the operation not only of the biogas converter 160, but also the aquatic plant cultivator 120, the anaerobic digester 140, and/or other associated systems and/or subsystems. Accordingly, the controller 168 can provide instructions to any of the components of the system 100 in an integrated fashion that improves the efficiency with which the overall system 100 produces the output methane 161.

In particular embodiments, the controller 168 can coordinate the operation of components of the system 100 to account for potential differences in the rates and modes with which the components operate. For example, the aquatic plant cultivator 120 may be active and solar-insolation heated during the day, and may be inactive or less active and cool at night, allowing the aquatic plants to rejuvenate. The anaerobic digester 140 may operate on a 24/7 schedule, but may be “fed” only periodically, e.g., once per day. The biogas converter 160 may also operate on a 24/7 schedule, but it and other components will periodically be shut down for service and/or maintenance. In a particular example, the aquatic plant cultivator 120 operates during the day, e.g., 12 hrs/day for approximately six months of the year, 8 hrs/day for approximately four months of the year and marginally for approximately two months of the year. The anaerobic digester 140 operates 24/7 and the biogas converter operates 24/7 for approximately 95% or more of the time. The conversion of MSW into biogas in the anaerobic digester 140 happens all year, so the quantities of input/output constituents can be scaled to adjust to the variation in aquatic plant biomass yields during the year. In addition to coordinating these varying rates and operation modes via the controller 168, the system 100 can include storage devices, and/or redundancies to smooth out rate differences among the components.

In a particular embodiment, the aquatic plant cultivator 120 can produce about 30 gm of aquatic plant biomass per day per square meter of open raceway pond. The average amount of biogas from a representative anaerobic digester 140 is expected to be about 0.5 liters[L]/gm of aquatic plant biomass. The resulting biogas production rate from the aquatic plant biomass is therefore expected to be about 0.53 scf biogas/d/m² of raceway pond surface. The addition of the MSW or other waste stream will increase the total biogas production accordingly. A small scale biogas converter 160 can convert about 0.96 MMscfd of digester biogas into about 5,000 gpd of high quality LBM/LNG for a conversion rate of about 192 scf biogas/gal LBM. This results in an overall production rate of about 0.002758 gpd LBM/m² [gallon of LBM per day per square meter]. A 1,000 acre raceway pond can accordingly produce about 11,161 gpd of LBM. The additional MSW or other waste stream increases the total production of LBM to over 20,000 gpd. The LBM can be stored in standard cryogenic tanks such as a 50,000 gallon tank and transported to fleet or other fuel customers via truck tankers as is widely done today.

In particular embodiments, the biogas provided to the prepurifier 162 can have a composition of about 65% methane, 32% carbon dioxide, 2% water, and 1% other constituents. The biogas can have approximately 1000 ppmv or less of noxious components, such as hydrogen sulfide, and can be provided at a temperature of about 90° F. and a pressure of about 2 psig. At the prepurifier 162, the sulfur level can be reduced to about 100 ppb, and the biogas can be compressed to about 125 psig. The water concentration can be reduced to 1 ppm, and trace volatile organic compounds can be removed. At the bulk purifier 163, the carbon dioxide concentration can be reduced to about 50 ppm, and the methane precooled. At the liquefier 164, the methane can be liquefied to form LNG/LBM, with about 99% methane. In other embodiments, the foregoing parameters can have other values, without departing from the scope of the present disclosure.

The foregoing arrangement can include a single system 100 (of the type shown in FIG. 1) or multiple systems 100. For example, a single system 100 can be employed to process biogas from about one square mile of land or section or 640 acres. The system can be placed centrally in this region, and can accordingly receive biogas from the four surrounding aquatic plant cultivators, each of which occupies about 160 acres. In other embodiments, the number of anaerobic digesters 140 and/or biogas converters 160 per unit area of aquatic plant cultivator can be different depending on factors including topography and system size. The activities of the single system 100 or multiple systems 100 can be controlled by the control module 168. Specific characteristics of a control module 168 in accordance with particular embodiments are described further below.

Many embodiments of the disclosure described above and described in further detail below may take the form of computer-executable instructions, including routines executed by a programmable computer, e.g., one or more components of the control module 168. Those skilled in the relevant art will appreciate that the disclosure can be practiced on a distributed control system (DCS) other than those shown and described below. The disclosure can be embodied in a special-purpose computer that is specifically programmed, configured or constructed to accept, record, and interpret numerous data inputs from multiple different temperature, pressure, flow rate, composition, and other transducers that provide information about all operational variables associated with the integrated plant 100. Information handled by these computers can be presented at any suitable display medium, including a CRT display or LCD. Representative computer systems for carrying out the processes described herein can include a SCADA (Supervisory Control and Data Acquisition) system.

Aspects of the disclosure can also be practiced in distributed environments, where tasks or modules are performed by remote processing devices that are linked through a communications network. In a distributed computing environment, program modules or subroutines may be located in local and remote memory storage devices. Aspects of the disclosure described below may be stored or distributed on computer-readable media, including magnetic or optically readable or removable computer disks, as well as distributed electronically over networks. Data structures and transmissions of data particular to aspects of the disclosure are also encompassed within the scope of the disclosure.

Representative Controllable Variables Associated with Aquatic Plant Cultivators

An aquatic plant cultivator configured in accordance with a representative embodiment, has operational constraints imposed by the duration and intensity of available sunlight. For example, aquatic plant growth may only occur for 8-12 hours per day, and aquatic plant harvesting conducted during a 6-8 hour process at night when the microalgae or other aquatic plant growth has sharply decreased. In another example, harvesting can be a continuous process during daylight hours. Introducing carbon dioxide into the aquatic plant cultivator may not be a continuous process but rather may be performed every few hours during the day and only once during the night, for example. To conduct the foregoing processes, multiple variables can be measured and used to provide appropriate control signals for various modules of equipment at the plant.

Representative variables include:
water circulation rate, which will typically be different for open and closed photobioreactor configurations;
sunlight intensity as a function of time and depth within the water;
water and air temperature, which will provide a basis for regulating the cooling or heating effect provided between the biogas converter 160 and other active modules;
wind velocities and relative humidity, which correlate with evaporation rates;
pH and level of water in the ponds, which will provide a basis for regulating pH adjustment and the flow of water from the anaerobic digester 140 into the aquatic plant cultivator 120;
concentration of microalgae or other aquatic plants in the aquatic plant cultivator 120 to control the flow rate of water with high concentrations of aquatic plant biomass into the collection ducts feeding into the anaerobic digester 140;
concentration of carbon dioxide in the water of the aquatic plant cultivator 120 at different locations in the circulation path to control the rate of carbon dioxide injection for optimal aquatic plant growth;
nutrient concentrations, e.g., for nitrogen and phosphorus, to control the flow of nutrient-loaded water from the anaerobic digester 140 back to the aquatic plant cultivator 120 for optimal aquatic plant growth; and
flow rates of water into the aquatic plant harvesting system going to/from the anaerobic digester 140.

In addition, unscheduled events such as heavy rain storms, very cold weather or other major disruptive events will impact the overall system 100 (e.g., the aquatic plant cultivator 120). Accordingly, the SCADA system or other controller 168 can be programmed to safely respond to potential consequences from such unpredictable events or infrequent equipment malfunctions. In particular embodiments, the controller 168 can direct the timing for transferring dry ice or liquid carbon dioxide to the aquatic plant cultivator 120 from the biogas converter 160 in response to a temperature sensor signal and/or a carbon dioxide sensor signal. The controller 168 can issue an instruction to an operator regarding the amount and timing of the dry ice or liquid carbon dioxide transfer. In particular embodiments, the system can include more automated transfer process (e.g., a conveyer belt) in which case the controller 168 can directly control the rate at which dry ice is conveyed to the aquatic plant cultivator 120.

### Anaerobic Digestion Plant

An anaerobic digester in accordance with a representative embodiment will produce biogas on a 24/7 basis, although several operational aspects of this plant will be conducted on a batch basis at appropriately scheduled intervals. For example, the process of transferring several types of waste streams from outside sources can be restricted to 8-10 daylight hours on week days, followed by grinding, sieving/sorting, blending, and sterilization in one or more holding tanks before the waste biomass is ready for mixing with the aquatic plant biomass prior to injection into the closed digester vessels. There are multiple variables in this portion of the plant that can be measured by sensors and processed by I/O panels to provide suitable inputs into the SCADA system.

Representative variables include:
digester temperature, e.g., to maintain suitable/optimal temperatures for anaerobic digester bacteria by controlling the amount of thermal energy used from the biogas converter 160;
time/temperature profiles at the holding tank 149 sufficient to kill the aquatic plants and destroy pathogens;
percentage of solids in each digester tank 146 to control the amount of fresh mixture to be pumped from the holding tank 149 to the various tanks;
biogas production flow rate, composition and temperature to control the rate of LBM production in the biogas converter 160;
variables associated with removal of the residual solids from the digester tanks 146 to one or more pressing/drying modules;
C/N ratio in the digester 140 to control blending of waste stream with aquatic plant biomass;
electrical loads from various equipment such as pumps, stirrers, heaters, instruments, etc. to control electrical demand from the genset at the biogas converter 160;
mass of external waste streams;
variables associated with grinding external waste streams into small particles suitable for rapid digestion;
stirring rate inside the digester tanks 146 to control the circulation pumps;
concentration of aquatic plants in the feedstock from the aquatic plant cultivator 120 to control the blending process with the other waste streams going into the sterilization holding tanks 149;
concentration of nitrogen and phosphorus in the return stream back to the aquatic plant cultivator 120 to optimize nutrient concentration for fast aquatic plant growth; and
levels of water in the digester tanks 146 to control how much is added with fresh biomass and how much is sent back to the aquatic plant cultivator 120.

### Biogas Converter

This portion of the system can operate on a 24/7 basis for ˜95% of the time. The biogas converter 160 typically requires much larger input power than the aquatic plant cultivator 120 and digester 140, and it produces a substantial amount of high grade waste energy. There are also several auxiliary systems such as instrument air, nitrogen, power for the plant, LBM storage tanks, and a cryogenic tanker transfer station that are integrated into the SCADA system for the purifier/liquefier system. Electrical power and thermal energy are available to the aquatic plant cultivator 120 for thermal management and other operational demands and to the anaerobic digester 140 for several operations. Representative examples of measured variables include:
electrical power demands from all portions of the system 100 to control the rate of electrical power production from the genset;
biogas flow rate from the digester 140 to control the capacity of the refrigeration module to match methane available;
wide range of temperatures, pressures, flow rates, and compositions to control the biogas converter 160;
level of liquid methane in the LBM storage tanks to coordinate the tankers used to transport the LBM from the plant to the end user;
flow rate of the waste heat gases along with the temperatures, pressures, and compositions of these gases to quantitatively control the supply of thermal energy to the aquatic plant cultivator 120 and anaerobic digester 140; and
parameters (e.g., quantity, timing, and delivery rate) for transferring dry ice or liquid carbon dioxide to the aquatic plant cultivator.
   The total number of instrumentation and control nodes in a typical landfill biogas-to-LNG plant is typically over 100, and a similar number of nodes is expected for each of the additional subsystems (e.g., the aquatic plant cultivator 120 and the digester 140) for internal operations.

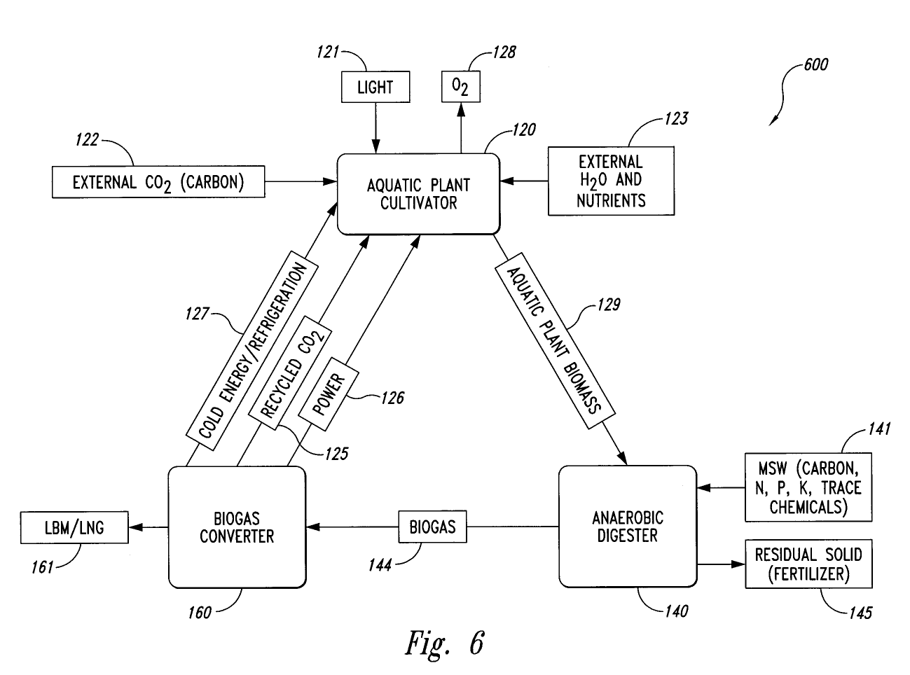

In other embodiments, certain aspects of the foregoing systems may be eliminated while still producing at least some of the benefits described above. For example, FIG. 6 illustrates a closed loop system in which the anaerobic digester 140 provides a reduced level of integration with the aquatic plant cultivator 120 and the biogas converter 160. In a particular aspect of this embodiment, the anaerobic digester 140 does not provide nutrients and water to the aquatic plant cultivator 120 and instead these constituents are provided externally, as indicated in block 123. The anaerobic digester 140 does not receive power or thermal energy from the biogas converter 160. Instead, the anaerobic digester 140 can have its own dedicated power source, and can receive thermal energy from other sources.

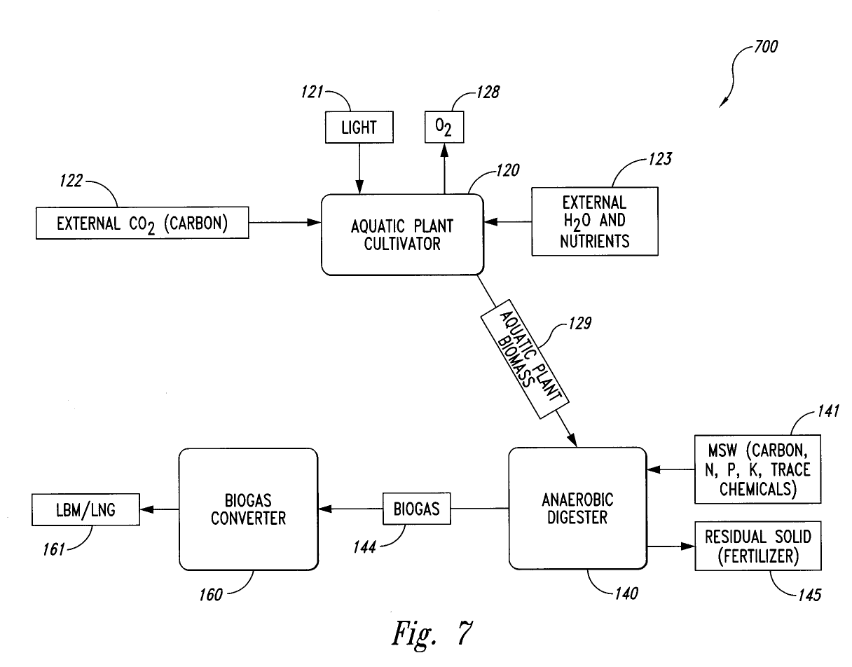

FIG. 7 illustrates still another example in which an overall system 700 operates in an open loop fashion. Accordingly, the aquatic plant cultivator 120 produces aquatic plant biomass 129, which is directed to the anaerobic digester 140. The anaerobic digester 140 produces biogas 144, which is directed to the biogas converter 160. The biogas converter 160 produces output methane 161. While this arrangement is not expected to be as efficient as the arrangements described above with reference to FIG. 5 and FIG. 6, it indicates that in particular embodiments, the system 700 can operate in an open loop fashion. In some instances, the system 700 may operate in an open loop fashion only during selected intervals, for example, when maintenance or other factors preclude the full or partial recyclable features described above with reference to FIGS. 1, 5 and 6.

One feature of several of the embodiments described above is that the aquatic plant cultivator, the anaerobic digester, and the biogas converter can be linked in a closed-loop fashion, and can include internal recycling and/or regeneration. This arrangement can synergistically improve the efficiency of the overall system beyond what might be available by merely improving the efficiencies of each of the individual components. In particular embodiments, the resulting system can produce LBM/LNG that is less expensive on an energy equivalent basis than diesel fuel, and provides a non-imported fuel which produces about 25% less carbon dioxide per mile when used as a transportation fuel, and produces much lower nitrogen oxide and particulate emissions. In addition, due to the internal recycling aspects of this arrangement, the carbon footprint of the system can be reduced when compared to comparable fuel production techniques. Accordingly, this system can provide a sustainable, renewable source of fuel, thus reducing the impact of the system on global warming, and reducing the need for importing fuels from other countries.

From the foregoing, it will be appreciated that specific embodiments of the invention have been described herein for purposes of illustration, but that various modifications may be made without deviating from the invention. For example, during certain phases of operation, aquatic plants from the aquatic plant cultivator may be used to produce foods, health supplements, omega-3 oils, chemicals, pharmaceuticals, pigments, biodiesel, and/or other constituents, in addition to or in lieu of providing biogas. Components of the system (e.g., the anaerobic digester and/or the biogas converter), may be made portable, as described above, and may be shipped from site to site (e.g., in standard containers). In other embodiments, these components may be permanently or semi-permanently located at a suitable site. The methane produced by the system can be used for transportation in some embodiments, and can have other end uses in other embodiments. The methane end product can be compressed, for example to extract or conserve cold energy used elsewhere in the system. Many of the parameters discussed above (e.g., concentrations, temperatures and flow rates) can have other values in other embodiments. While several arrangements for internally recycling energy and constituents were described above in the context of FIGS. 1-7, systems in accordance with other embodiments can include other arrangements for recycling the same and/or different constituents and/or energy forms. Several embodiments described above were described in the context of batch processes and associated systems. In other embodiments, similar or identical results may be obtained via continuous flow processes and systems.

Certain aspects of the invention described in the context of particular embodiments may be combined or eliminated in other embodiments. For example, multiple systems 100 having components similar to those shown in FIG. 1 can be linked in an overall system. Further, while advantages associated with certain embodiments of the invention have been described in the context of those embodiments, other embodiments may also exhibit such advantages, and not all embodiments need necessarily exhibit such advantages to fall within the scope of the invention.

I/We claim:

1. A system for processing methane, comprising:
an aquatic plant cultivator;
an anaerobic digester operatively coupled to the aquatic plant cultivator to receive aquatic plants and produce biogas;
a biogas converter coupled to the anaerobic digester to receive the biogas and produce liquefied methane and thermal energy, at least a portion of the thermal energy resulting from a methane liquefaction process;
a thermal path between the biogas converter and at least one of the aquatic plant cultivator and the anaerobic digester; and
a controller coupled to the biogas converter and the at least one of the aquatic plant cultivator and the anaerobic digester, the controller being programmed with instructions that, when executed, direct the portion of thermal energy between the biogas converter and the at least one of the aquatic plant cultivator and the anaerobic digester.

2. The system of claim 1 wherein the biogas converter includes a refrigeration cycle, and wherein the thermal path is positioned to transmit a refrigerated substance from the biogas converter to the aquatic plant cultivator.

3. The system of claim 2 wherein the refrigerated substance includes dry ice and wherein the thermal path is positioned to transfer the dry ice to the aquatic plant cultivator.

4. The system of claim 1 wherein the biogas converter includes a refrigeration cycle, and wherein the portion of the thermal energy includes thermal energy produced by the refrigeration cycle.

5. The system of claim 1 wherein the thermal path includes a first portion connected between the biogas converter and the aquatic plant cultivator, and a second portion connected between the biogas converter and the anaerobic digester.

6. The system of claim 1 wherein the anaerobic digester is coupled to the aquatic plant cultivator to return anaerobic digester by-products to the aquatic plant cultivator.

7. The system of claim 1, further comprising:
an aquatic plant biomass path coupled between the aquatic plant cultivator and the anaerobic digester to direct an aquatic plant biomass to the anaerobic digester;
an anaerobic digester return path coupled between the anaerobic digester and the aquatic plant cultivator to direct output from the anaerobic digester to the aquatic plant cultivator;
a biogas path coupled between the anaerobic digester and the biogas converter to direct biogas to the biogas converter; and wherein the thermal path includes:
a first thermal return path coupled between the biogas converter and the anaerobic digester to direct a first thermal output from biogas converter to the anaerobic digester;
a second thermal return path coupled between the biogas converter and the aquatic plant cultivator to direct a second thermal output from biogas converter to the aquatic plant cultivator; and wherein
the controller is coupled to the aquatic plant cultivator, the anaerobic digester, and the biogas converter, the controller being programmed with instructions that, when executed, direct flows of constituents and energy among the aquatic plant cultivator, the anaerobic digester, and the biogas converter.

8. The system of claim 7 wherein the anaerobic digester return path carries at least one of nutrients and water from the anaerobic digester to the aquatic plant cultivator.

9. The system of claim 8, further comprising a municipal solid waste path coupled to the anaerobic digester to provide municipal solid waste to the anaerobic digester.

10. The system of claim 9 wherein the anaerobic digester includes:
an anaerobic digester vessel;
a holding vessel positioned to receive the municipal solid waste via the municipal solid waste path, and receive aquatic plants from the aquatic plant cultivator;
a pre-processor positioned to receive a mixture of aquatic plants and municipal solid waste from the holding vessel, and control a moisture content of the mixture;
a flow path coupled between the pre-processor and the anaerobic digester vessel to convey the mixture from the pre-processor to the anaerobic digester vessel;
a first heat exchanger positioned to heat aquatic plants upstream of the holding vessel;
a second heat exchanger positioned between the holding vessel and the pre-processor to cool the mixture entering the pre-processor; and
a fluid flow path coupled between the pre-processor and the aquatic plant cultivator to transfer waste liquid from the pre-processor to the aquatic plant cultivator, the fluid flow path passing through the second heat exchanger to cool the mixture entering the pre-processor, the fluid flow path passing through the first heat exchanger to heat the aquatic plants entering the holding vessel.

11. A system for processing methane, comprising:
an aquatic plant cultivator;
an anaerobic digester operatively coupled to the aquatic plant cultivator to receive aquatic plants and produce biogas;
a biogas converter coupled to the anaerobic digester to receive the biogas and produce liquefied methane and carbon dioxide at a temperature different than a temperature at the aquatic plant cultivator;
a thermal path between the biogas converter and the aquatic plant cultivator; and
a controller coupled to the biogas converter and the aquatic plant cultivator, the controller being programmed with instructions that, when executed, identify parameters for transferring the carbon dioxide from the biogas converter to the aquatic plant cultivator.

12. The system of claim 11 wherein the controller is programmed with instructions directing the timing for transferring carbon dioxide from the biogas converter to the aquatic plant cultivator.

13. The system of claim 11 wherein the controller is programmed with instructions directing the physical conveyance of carbon dioxide from the biogas converter to the aquatic plant cultivator.

14. The system of claim 11 wherein the controller is programmed with instructions directing the transfer of waste heat from the biogas converter to the aquatic plant cultivator to heat the aquatic plant cultivator.

15. The system of claim 11 wherein the controller is operatively coupled to a carbon dioxide sensor at the aquatic plant cultivator, and wherein the instructions include directing a transfer of carbon dioxide to the aquatic plant cultivator in response to a signal from the carbon dioxide sensor corresponding to a low carbon dioxide level.

16. The system of claim 11 wherein the controller is operatively coupled to a temperature sensor at the aquatic plant cultivator, and wherein the instructions include directing a transfer of cold carbon dioxide to the aquatic plant cultivator in response to a signal from the temperature sensor corresponding to a high temperature.

17. The system of claim 16 wherein the cold carbon dioxide includes dry ice, and wherein the thermal path includes a dry ice conveyance device.

18. A system for processing methane, comprising:
an aquatic plant cultivator;
an anaerobic digester operatively coupled to the aquatic plant cultivator to receive aquatic plants and municipal solid waste and produce biogas;
a biogas converter coupled to the anaerobic digester to receive the biogas and produce liquefied methane and thermal energy;
a thermal path between the biogas converter and the anaerobic digester; and
a controller coupled to the biogas converter and the anaerobic digester, the controller being programmed with instructions that, when executed, direct thermal energy from the biogas converter to the anaerobic digester to heat the aquatic plants and the municipal solid waste.

19. The system of claim 18 wherein the anaerobic digester includes an anaerobic digestion vessel, and wherein the thermal path is operatively coupled to the anaerobic digestion vessel to heat the aquatic plants and the municipal solid waste to a temperature suitable for anaerobic digestion.

20. The system of claim 18 wherein the anaerobic digester includes an anaerobic digestion vessel and a holding vessel coupled to the anaerobic digestion vessel to provide constituents to the anaerobic digestion vessel, and wherein the thermal path is positioned to heat constituents that are at least one of (a) upstream of the holding vessel, (b) in the holding vessel, or (c) in the anaerobic digestion vessel.

21. The system of claim 18 wherein the biogas converter is further coupled to the anaerobic digester to provide electrical power to the anaerobic digester.

22. The system of claim 18 wherein the thermal energy includes waste heat from a refrigeration cycle at the biogas converter.

23. A system for processing methane, comprising:
an aquatic plant cultivator;
a pre-treatment device operatively coupled to the aquatic plant cultivator and a source of municipal solid waste (MSW), the pre-treatment device having a heat exchanger positioned to heat aquatic plants and the MSW;
an anaerobic digester vessel operatively coupled to the pre-treatment device to receive the aquatic plants and MSW and produce biogas;
a biogas converter coupled to the anaerobic digester vessel to receive the biogas and produce liquefied methane and thermal energy, at least part of the thermal energy being produced by a refrigeration cycle, the thermal energy including thermal energy stored in carbon dioxide;
an anaerobic digester return path coupled between the anaerobic digester vessel and the aquatic plant cultivator to direct output from the anaerobic digester vessel to the aquatic plant cultivator;
a biogas path coupled between the anaerobic digester vessel and the biogas converter to direct biogas to the biogas converter;
a first biogas converter return path coupled between the biogas converter and at least one of the pre-treatment device and the anaerobic digester vessel to direct a first thermal output from the biogas converter to the at least one of the pre-treatment device and the anaerobic digester vessel;
a second biogas converter return path coupled between the biogas converter and the aquatic plant cultivator to direct a second thermal output from biogas converter to the aquatic plant cultivator, the second thermal output including the carbon dioxide; and
a controller coupled to the aquatic plant cultivator, the pretreatment device, the anaerobic digester vessel, and the biogas converter, the controller be programmed with instructions that, when executed, direct flows of energy and constituents among the aquatic plant cultivator, the pretreatment device, the anaerobic digester vessel, and the biogas converter.

24. The system of claim 23 wherein the controller is programmed with instructions that direct the carbon dioxide to the aquatic plant cultivator in response to an indication of low carbon dioxide at the aquatic plant cultivator.

25. The system of claim 23 wherein the controller is programmed with instructions that direct the carbon dioxide to the aquatic plant cultivator in response to an indication of high temperature at the aquatic plant cultivator.

26. A method for processing methane, comprising:
growing aquatic plants at an aquatic plant cultivator;
receiving the aquatic plants at an anaerobic digester;
producing biogas at the anaerobic digester;
receiving the biogas at a biogas converter;
liquefying methane from the biogas at the biogas converter;
producing at least a portion of thermal energy at the biogas converter as a result of liquefying the methane; and
transferring the portion of thermal energy to at least one of the anaerobic digester and the aquatic plant cultivator.

27. The method of claim 26, further comprising:
automatically monitoring a rate of aquatic plant production at the aquatic plant cultivator;
automatically monitoring a rate of biogas production at the anaerobic digester;
automatically monitoring a rate of liquid methane production at the biogas converter; and
automatically controlling a flow of energy and materials among the aquatic plant cultivator, the anaerobic digester and the biogas converter based at least in part on the rate of aquatic plant production, the rate of biogas production, and the rate of liquid methane production.

28. The method of claim 26 wherein growing aquatic plants includes growing microalgae.

29. The method of claim 26 wherein growing aquatic plants includes growing duckweed.

30. The method of claim 26 wherein transferring the portion of thermal energy includes transferring refrigeration energy to the aquatic plant cultivator.

31. The method of claim 26 wherein transferring the portion of thermal energy includes transferring dry ice or cold liquid carbon dioxide to the aquatic plant cultivator.

32. The method of claim 26 wherein transferring the portion of thermal energy includes transferring thermal energy that is not a direct result of combusting biogas or liquefied methane.

33. The method of claim 26, wherein the anaerobic digester includes a holding vessel, a pre-treatment device coupled to the holding vessel, and an anaerobic digester vessel coupled to the pre-treatment device, and wherein the method further comprises:
directing aquatic plants from the aquatic plant cultivator into the holding vessel;
directing municipal solid waste into the holding vessel;
carrying a mixture of the aquatic plants and the municipal solid waste in the holding vessel at an elevated temperature to kill at least a portion of the aquatic plants and kill pathogens in the mixture;
increasing a solids fraction of the mixture by removing liquid from the mixture at the pre-treatment device;
directing the mixture to the anaerobic digester vessel;
directing the liquid removed from the mixture through a first heat exchanger and a second heat exchanger;
at the second heat exchanger, transferring heat from the removed liquid to the mixture entering the pre-treatment device;
at the first heat exchanger transferring heat from the removed liquid to the aquatic plants entering the storage vessel; and
returning at least a portion of the removed liquid to the aquatic plant cultivator.

34. The method of claim 26, wherein the anaerobic digester includes a holding vessel, a pre-treatment device coupled to the holding vessel, and an anaerobic digester vessel coupled to the pre-treatment device, and wherein the method further comprises:
removing water and nutrients from the anaerobic digester vessel;
transferring heat from the removed water and nutrients to aquatic plants entering the holding vessel; and
carrying the aquatic plants in the holding vessel at an elevated temperature to kill at least a portion of the aquatic plants.

35. The method of claim 26, wherein the anaerobic digester includes a holding vessel, a pre-treatment device coupled to the holding vessel, and an anaerobic digester vessel coupled to the pre-treatment device, and wherein the method further comprises:
directing an aquatic plant-containing flow from the holding vessel to the pre-treatment device;
removing fluid from an aquatic plant-containing flow at the pre-treatment device;
pre-heating the aquatic plant-containing flow entering the pre-treatment device with removed fluid from the pre-treatment device;
pre-heating aquatic plants entering the holding vessel with removed fluid from the pre-treatment device; and
directing the removed fluid to the aquatic plant cultivator.

36. A method for processing methane, comprising:
growing aquatic plants at an aquatic plant cultivator;
receiving the aquatic plants at an anaerobic digester;
producing biogas at the anaerobic digester;
receiving the biogas at a biogas converter;
liquefying methane from the biogas at the biogas converter;
producing carbon dioxide at the biogas converter; and
transferring the carbon dioxide to the aquatic plant cultivator.

37. The method of claim 36 wherein growing aquatic plants includes growing microalgae.

38. The method of claim 36 wherein growing aquatic plants includes growing duckweed.

39. The method of claim 34 wherein producing carbon dioxide includes producing solid or liquid carbon dioxide as a by-product of liquefying the methane.

40. The method of claim 34 wherein transferring the carbon dioxide to the aquatic plant cultivator includes transferring the carbon dioxide to cool the aquatic plants at the aquatic plant cultivator in response to an indication that a temperature at the aquatic plant cultivator is above a target value.

41. The method of claim 34 wherein transferring the carbon dioxide to the aquatic plant cultivator includes transferring the carbon dioxide in response to an indication that a carbon dioxide level at the aquatic plant cultivator is below a target value.

42. The method of claim 34, further comprising directing heat resulting from liquefying the methane at the biogas converter, to the aquatic plant cultivator in response to an indication that a temperature at the aquatic plant cultivator is below a target value.

43. The method of claim 34 wherein transferring the carbon dioxide includes transferring carbon dioxide at a temperature higher than a temperature at the aquatic plant cultivator.

44. The method of claim 34, further comprising:
automatically monitoring a rate of aquatic plant production at the aquatic plant cultivator;
automatically monitoring a rate of biogas production at the anaerobic digester;
automatically monitoring a rate of liquid methane production at the biogas converter; and
automatically controlling a flow of energy and materials among the aquatic plant cultivator, the anaerobic digester and the biogas converter based at least in part on the rate of aquatic plant production, the rate of biogas production, and the rate of liquid methane production.

45. A method for processing methane, comprising:
growing a first portion of aquatic plants at an aquatic plant cultivator;
directing the first portion of the aquatic plants and a first portion of municipal solid waste (MSW) to a pre-treatment device;
heating the first portion of the aquatic plants and the first portion of the MSW at the pre-treatment device to kill the first portion of the aquatic plants and pathogens carried by at least one of the first portion of the aquatic plants and the first portion of the MSW;
directing the first portion of the aquatic plants and the first portion of the MSW to an anaerobic digester vessel to produce biogas;
directing the biogas from the anaerobic digester vessel to a biogas converter to produce liquefied methane and thermal energy; and
directing at least a portion of the thermal energy from the biogas converter to the pre-treatment device to kill a second portion of the aquatic plants and pathogens carried by at least one of the second portion of the aquatic plants and a second portion of the MSW.

46. The method of claim 41 wherein the pre-treatment device includes a holding vessel and wherein directing thermal energy includes directing thermal energy to the aquatic plants before they enter the holding vessel.

47. The method of claim 41 wherein the pre-treatment device includes a holding vessel and wherein directing thermal energy includes directing thermal energy to aquatic plants in the holding vessel.

48. The method of claim 41 wherein the portion of thermal energy is a first portion, and wherein the method further comprises directing at least a second portion of the thermal energy to the anaerobic digester vessel.

49. The method of claim 41, further comprising:
removing liquid from the first portion of the aquatic plants and the first portion of the MSW before directing the first portions to the anaerobic digester vessel; and
transferring heat from the removed liquid to heat at least one of the second portion of the aquatic plants and the second portion of MSW.

50. A method for processing methane, comprising:
growing aquatic plants by receiving sunlight, recycled carbon dioxide, recycled water and recycled nutrients at an aquatic plant cultivator;
receiving the aquatic plants and municipal solid waste (MSW) at a pre-treatment device;
directing recycled thermal energy to the pre-treatment device to kill the aquatic plants and kill pathogens carried by at least one of the aquatic plants and the MSW;
directing the aquatic plants and the MSW from the pre-treatment device to an anaerobic digester vessel;
producing nutrients, water and biogas at the anaerobic digester vessel;
recycling the nutrients and water by directing the nutrients and water to the aquatic plant cultivator;
receiving the biogas at a biogas converter;
liquefying methane from the biogas at the biogas converter;
producing carbon dioxide from the biogas at the biogas converter;
producing waste heat at the biogas converter as a result of liquefying the methane;
recycling the carbon dioxide and controlling a temperature at the aquatic plant cultivator by directing the carbon dioxide to the aquatic plant cultivator;
recycling a first portion of the waste heat by directing the first portion to the aquatic plant cultivator;
recycling a second portion of the waste heat by directing the second portion to at least one of the pre-treatment device and the anaerobic digester vessel.

51. The method of claim 46 wherein the pre-treatment device includes a holding vessel and a pre-processor, and wherein the method further comprises:
holding a mixture of the aquatic plants and the MSW at an elevated temperature in the holding vessel;
directing the mixture to the pre-processor;
removing liquid from the mixture at the pre-processor;
transferring heat from the removed liquid to a portion of the mixture entering the pre-processor; and
transferring heat from the removed liquid to a portion of the aquatic plants entering the holding vessel.
